(19) United States
(12) Patent Application Publication
(10) Pub. No.: US 2012/0101471 A1
(43) Pub. Date: Apr. 26, 2012

(54) SELF-POWERED INFUSION DEVICE AND METHOD OF USE

(76) Inventors: Giorgio di Palma, West Valley City, UT (US); William A. Cartier, Glenville, NY (US)

(21) Appl. No.: 13/279,427

(22) Filed: Oct. 24, 2011

Related U.S. Application Data
(60) Provisional application No. 61/405,864, filed on Oct. 22, 2010.

(51) Int. Cl. A61M 5/145; A61M 5/142

(52) U.S. Cl. 604/500; 604/151

(57) ABSTRACT

A medical device system is provided here which has a disposable pump member capable of delivering fluid at a high pressure and a controlled rate. The disposable pump member has an internal power source that provides an energy source for a drivable pump used during treatment. Additionally, a method is provided herein for infusing, injecting, or delivering an intended fluid into a body vessel at a high pressure and a controlled rate.

### CROSS REFERENCE TO RELATED APPLICATIONS

This application claims priority under 35 U.S.C. §119(e) to U.S. Provisional Application No. 61/405,864, filed Oct. 22, 2010, which is incorporated herein by reference.

### TECHNICAL FIELD

The present invention relates generally to devices for delivering fluids, drugs or other medical preparations to a site within a patient's body. More specifically, the invention relates to a self-powered infusion device that pumps fluids, drugs or other medical preparations at controlled pressure and rate through a delivery means into a lumen of a blood vessel or another cavity or lumen within a patient's body. A method of use is also disclosed.

### BACKGROUND OF THE INVENTION

Veins are thin-walled and contain one-way valves that control blood flow. Normally, the valves open to allow blood to flow into the deeper veins and close to prevent back-flow into the superficial veins. When the valves are malfunctioning or only partially functioning, however, they no longer prevent the back-flow of blood into the superficial veins. As a result, venous pressure builds at the site of the faulty valves. Because the veins are thin walled and not able to withstand the increased pressure, they become what are known as varicose veins which are veins that are dilated, tortuous or engorged.

In particular, varicose veins of the lower extremities are one of the most common medical conditions of the adult population. It is estimated that varicose veins affect approximately 25% of adult females and 10% of males. Symptoms include discomfort, aching of the legs, itching, cosmetic deformities, and swelling. If left untreated, varicose veins may cause medical complications such as bleeding, phlebitis, ulcerations, thrombi and lipodermatosclerosis.

Traditional treatments for varicosities include both temporary and permanent techniques. Temporary treatments involve use of compression stockings and elevation of the diseased extremities. While providing temporary relief of symptoms, these techniques do not correct the underlying cause that is the faulty valves. Permanent treatments include surgical excision of the diseased segments, ambulatory phlebectomy, and occlusion of the vein through thermal means.

Minimally invasive thermal treatments for venous insufficiency eliminate the need for general anesthesia and have relatively short recovery times. Endovascular thermal energy therapy is a relatively new treatment technique for venous reflux diseases. With this technique, thermal energy in the form of laser or radio frequency (RF) energy is delivered by an energy delivery device that is percutaneously inserted into the diseased vein prior to energy delivery. In a laser therapy, an optical fiber is used as the energy delivery device whereas in an RF therapy, RF electrodes are used as the energy delivery device.

Chemical occlusion, also known as sclerotherapy, is an in-office procedure involving the injection of an irritant chemical directly into the vein. The drug is delivered either through direct injections with a small gauge needle or more recently using a catheter placed in the target vein. The chemical acts upon the inner lining of the vein walls causing them to occlude and block blood flow. The use of liquid sclerosing agents to treat varicosities has been utilized for decades, but has traditionally been limited to veins with diameters less than 5 mm.

Sclerotherapy to treat larger diameter veins has not been widely used due mainly to recommended volume limit of the drug and reported failure rates. Sufficient drug must be delivered to the treatment zone to fully displace the blood. A typical sclerosant, such as 3% sodium tetradecyl sulfate, is volume limited to a maximum of 10 cc per treatment, making it difficult to treat larger veins. Catheter-directed sclerotherapy has been attempted in larger veins such as the Great Saphenous Vein using liquid sclerosant. Although initially successful, long-term failure rates are reportedly high, due to inadequate concentrations of drug being delivered to the vessel to cause durable closure and permanent destruction of the vein. It is also postulated that blood flow in larger veins prevents the sclerosant from reaching the vessel wall in sufficient concentration to effectively destroy the inner vessel wall lining to occlude the vessel, resulting in a relatively low treatment success rate.

To minimize the dilution of the agent by blood, some practitioners have utilized methods of emptying as much blood volume as possible from the vein being treated. Vein emptying may be performed by placing the patient in a Trendelenberg position with the target leg higher than the torso. Emptying may also be facilitated by the use of manual compression using either compression bandages or finger compression at the proximal and distal ends of the vein. These techniques, while lowering the overall blood volume in the vessel, are time-consuming, require additional personnel to maintain compression during the procedure, are uncomfortable to the patient, and often result in incomplete blood removal and inconsistent treatment results.

The self-powered infusion device allows users to deliver a controlled and precise amount of fluid at high pressures through a catheter to the treatment site. For example, current sclerotherapy treatments are done with manual delivery using a syringe that can lead to varied dispersion pressures during treatment resulting in unequal and inconsistent delivery of the fluid or drug during treatment. This device is an improvement on current sclerotherapy treatment because the self-powered infusion device can infuse a precise volume of drug, fluid or sclerosing agent, such as Sotradecol® sclerosing agent, at a controlled pressure, rate, and dispersion patterns which can all be delivered uniformly during a treatment session. As used herein, a sclerosant refers to any fluid that acts upon the inner vessel wall causing a diameter reduction and subsequent vessel wall destruction and occlusion. Sclerosant fluid as defined herein may be liquid in varying concentrations. The sclerosant may also be combined with other adjunctive drugs such as vaso-spasming fluid, anesthetics, saline or other fluids such as tumescent anesthesia.

### SUMMARY

The invention is a self-powered infusion pump for delivering fluid at a high pressure and controlled rate. The invention comprises of a disposable pump housing, an inlet channel in fluid communication with an inlet valve for selectively allowing fluid to flow between a fluid supply and a pump chamber. The pump chamber comprises of a dome reservoir and convex dome diaphragm being resiliently biased towards a selected first cup shape and first volume that is moveable towards a second depressed shape and second volume, an outlet valve for selectively allowing fluid to flow between the dome reservoir and an outlet.

The disposable pump member of the self-powered infusion device is intended to be disposed of after each use. The disposable pump member of the device contains a disposable power source which powers a drive mechanism that creates the necessary pressure levels to infuse fluid. The disposable power source also allows the user to more easily complete treatment the device because it does not require external connections or recharging between uses.

A method of infusing fluid at a controlled high pressure using a self-powered infusion pump is described herein. The method begins with inserting a disposable pump housing into a delivery device. Next, the user places/connects a fluid source to the inlet and powers the motor of the delivery device with the disposable power source. The disposable power source drives a plunger compressing the dome towards a second depressed shape and a lower volume reservoir capacity. After the plunger returns to its original state the dome returns to the first shape and first volume, causing an increase in internal pressure as the fluid travels though the inlet valve and fills the dome reservoir. Finally, the user powers the motor with the disposable power source and compresses the dome reservoir with the plunger towards the second depressed shape and volume forcing the fluid through an outlet valve and exit, thus delivering fluid at a high pressure and controlled rate.

Currently, the most advantageous method of delivering a sclerosant agent to the inner vein wall during treatment is with minimal or no dilution by the blood. This device will enable the user to control the infusion rate or pressure of the drug or sclerosant fluid during treatment and the device will be used to repeatedly infuse or deliver the drug or sclerosant fluid at high pressure and high velocity. The result of delivering a sclerosing agent with high velocity is to provide a force capable of penetrating through the blood to contact the vessel wall. If additional sclerosant infusions are desired to ensure complete wall coverage the device is capable of repeating the same controlled infusion rates. Therefore, this invention will allow for the scerlosing agent the be delivered to the vein wall with minimal dilution from blood and have a similar wall vein wall coverage to a foam sclerosing agent but without the negative side effects associated with a foam agent. For example, the use of a foam sclerosant agent may cause small air bubbles that can travel within the patient's vasculature to the brain or other organs. The results of these negative side effects may be severe damage to the organs or even patient death.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a plan view of the self-powered infusion device.

FIG. 2 is an isometric view of the disposable member of the self-powered infusion device.

FIG. 3 is a partial enlarged cross-sectional view of the disposable pump member of the self-powered infusion device illustrating fluid flow through the device in one embodiment of the disposable member.

FIG. 4 is a partial enlarged cross-sectional view of the reservoir of the disposable pump member.

FIG. 5 is a cross-sectional view of the self-powered infusion device

FIG. 6 is an isometric view of the disposable member of the self-powered infusion device illustrating a second embodiment of the disposable member.

FIG. 7 is a partial enlarged cross-sectional view of the disposable member of the self-powered infusion device illustrating a second embodiment.

FIG. 8 is a cross-sectional view of the self-powered infusion device illustrating a second embodiment.

FIG. 9 is a flow chart for the method of use.

### DETAILED DESCRIPTION OF THE INVENTION

The present invention can be understood more readily by reference to the following detailed description and the examples included therein and to the Figures and their previous and following description. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein is a fluid delivery and treatment device intended for the delivery of fluids with an anatomic lumen or cavity and treatment of the desired region.

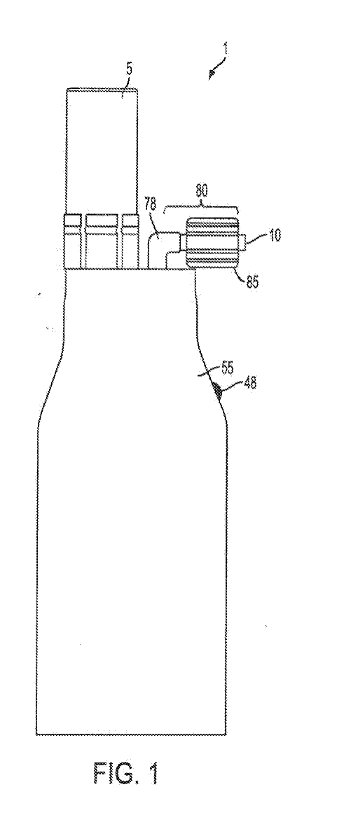

Referring to FIG. 1, a self-powered delivery device 1 for controlling the pressure of infusing material into the vascular system or other tubular anatomical structure is provided herein. The purpose of controlling the pressure and rate of infusing material, such as a sclerosant agent, into the vascular system is to prevent blood dilution of the material, properly deliver infusion material to a target area, and provide the user with precise control over the pressure and rate of delivery of the infusion material. For example, when treating a varicosed vein with sclerosant it is critical to deliver the drug directly to the vein wall itself rather than directing the sclerosant in the vessel lumen where it is diluted by the blood flow. By infusing a sclerosant agent through the delivery device 1 at a controlled high pressure and high delivery rate, the sclerosant agent is not diluted by blood which allows for a greater concentration of sclerosant agent to be delivered to the vein wall; both of which will shorten the procedure time and decrease the amount of sclerosant needed to complete the procedure. The delivery device 1 is comprised of a reusable drive mechanism 55, a pump member 50, and a fluid source 5. The pump member 50 comprises of an inlet means 75, a resilient diaphragm member 35, and an outlet means 80 which further comprises an outlet port 10 and a connection hub 85 used to connect the delivery device 1 to a delivery means (not shown) such as a catheter. The delivery device 1 may be sized to be compact and small enough to fit in one hand of the user during use.

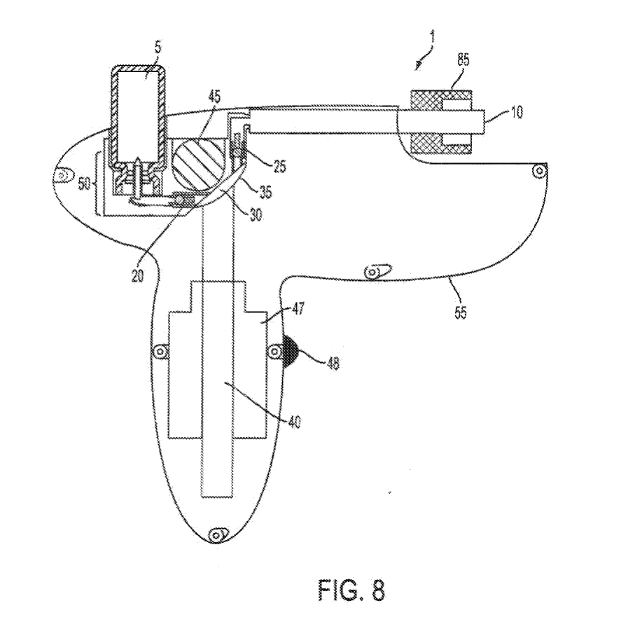

The purpose of the disposable pump member 50 is to provide a fluid path for the delivery of fluid from the fluid source 5 to the delivery catheter, which in turn will convert fluid pressure from low pressure to high pressure and high velocity. In one embodiment, the pump member 50 may be removable and may be replaced after each use. The pump member 50 is designed to be connected to and placed in the drive mechanism 55. The disposable pump member 50 may be connected to the drive mechanism 55 in a number of different ways depending on the shape of the drive mechanism 55. For example, FIG. 1 illustrates the disposable pump member 50 being inserted, slid into, or attached to the top of the drive mechanism 55. However, other attachment configurations between the disposable pump member 40 and drive mechanism 55 are shown in FIG. 8, and are within the scope of this invention. The fluid source 5 is attached to the disposable pump member 50. The fluid source 5 is comprised of a container to house fluid such as a medical vial or syringe. The fluid source 5 contains the fluid, medicine, or drug that is being delivered. Examples of the types of fluids include a sclerosing agent, chemotherapeutic or ablative agents, thrombolytic agents, antibiotics, saline, heparin, anesthesia, such as tumescent anesthesia, or other treatment fluids. The fluid source 5 can take a number of different shapes and sizes, such as a syringe, vial, intravenous therapy bag, or standard disposable tubing known in the art. In this embodiment, the fluid source 5 is in the shape of a vial.

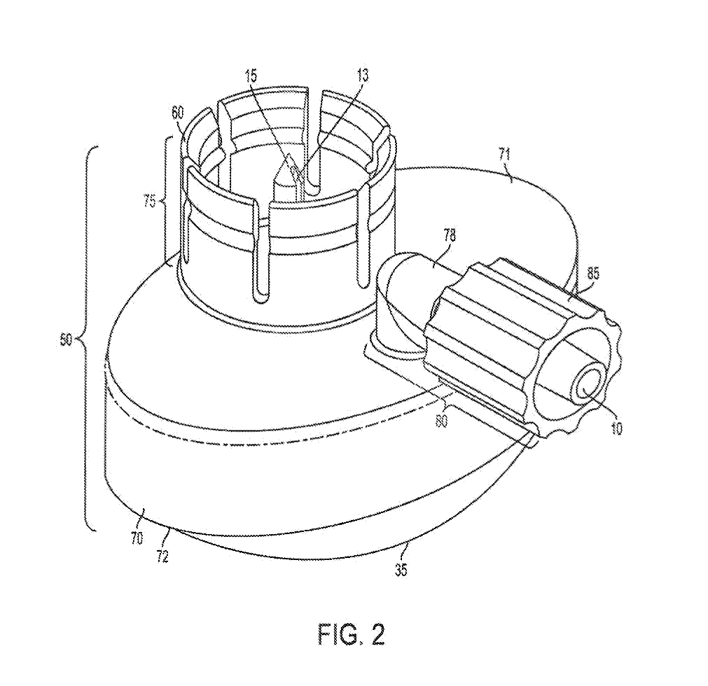

FIG. 2 is an isometric view of one embodiment of the disposable pump member 50 of the self-powered infusion device. The disposable pump member 50 is comprised of a housing 70 having a top surface 71 and bottom surface 72. The top surface 71 of the housing 70 contains both an inlet means 75 and an outlet means 80. The inlet means 75 provides an inlet fluid channel for fluid from the fluid source 5 (FIG. 1) to enter the disposable pump member 50. The outlet means 80 provides an exit fluid channel for the pressurized fluid to flow from the disposable pump member 50 into the delivery mechanism (not shown), such as a catheter. The bottom surface 72 of the housing 70 contains a diaphragm member 35. The empty space between the diaphragm member 35 and bottom surface 72 of the housing 70 comprises the reservoir (not shown in FIG. 2). Because the pump member 50 is disposable, each component of the pump member 50 may be manufactured from a number of different plastic materials or any other lightweight, durable, disposable materials. The outer profile of the pump member 50 may vary depending on the type of drive mechanism 55 used.

The inlet means 75 may comprise of a connection member 60 and a piercing member 15 which includes an inlet channel 13. The connection member 60 is used to attach onto and secure the fluid source 5. The connection member 60 can be any of the standard luer connections as known in the art, and in this embodiment the connection member 60 is a female luer. The connection member 60, piercing member 15 and inlet channel 13 may all protrude in an upward direction away from the top surface 71 of the housing 70. However, in an alternative embodiment it is also conceivable that the connection member 60 may be in the form of an indwelling or indent in the top surface 71 of the housing 70 and does not extend in an upward direction. Furthermore, in this alternative embodiment the fluid source 5 would securely fit or lock within this indwelling or indent of the connection member 60.

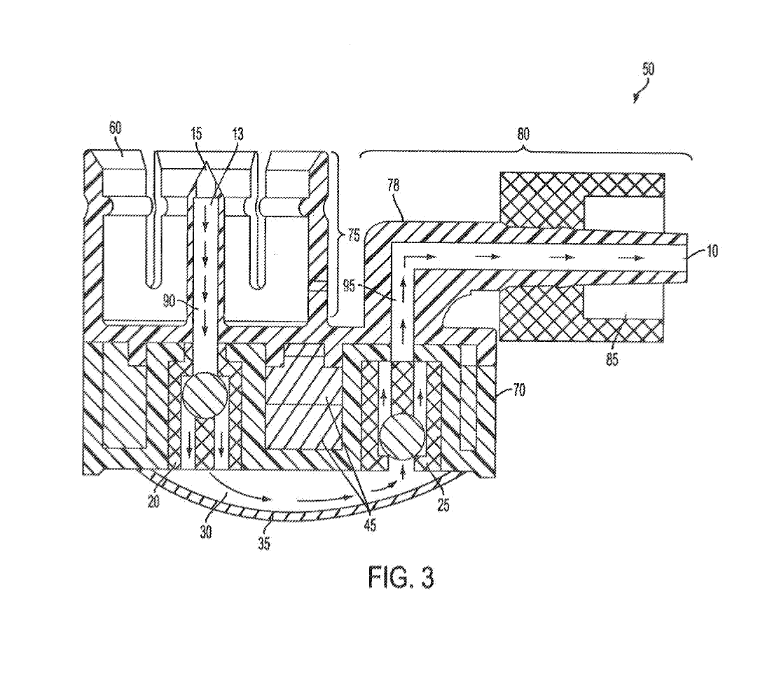

The inlet channel 13 has an opening that allows fluid to enter into the first fluid communication channel 90 (as seen in FIG. 3). If a vial is used as the fluid source 5 then as the user inserts the fluid source 5 into the connection member 60 this will cause the piercing member 15 to pierce through the septum of the vial. Alternatively, if using a standard syringe as the fluid source 5 then this piercing action may not be necessary when establishing a fluid pathway from the syringe to the disposable pump member 55 via the inlet channel 13. Once the piercing member 15 is positioned within the fluid source 5 open fluid communication may be created between the fluid source 5 and the inlet channel 13.

The outlet means 80 is in open fluid communication with the second fluid communication channel 95 (as seen in FIG. 3) and contains an elbow 78, outlet port 10, and a connection hub 85. In this embodiment, the outlet means 80 extends upward from the top surface 71 of the housing 70. The purpose of the outlet means 80 is to securely connect the device to a delivery means (not shown), such as an infusion, catheter. The outlet means may be connected to the proximal most end of the selected delivery means. The connection between the connection hub 85 and proximate end of the selected delivery means may be that of a common luer connection which is known in the art.

The diaphragm 35 may be made of a resilient waterproof material, such as a plastic, rubber, silicone, or other materials known in the art. When assembled, the diaphragm 35 forms a water proof seal against the bottom surface 72 of the housing 70. The diaphragm 35 may be connected to the bottom surface 72 of the housing 70 by a number of different attachment means known in the art, including but not limited to the use of adhesive, an o-ring, or other bracket attachment means. The diaphragm 35 is resiliently biased to its point of lowest potential energy, or a relaxed state, in which it forms a cup, convex or dome shape extending away from the housing 70. Because the diaphragm 35 is made of resilient material it also has a compressed state (not shown). The compressed state of the diaphragm member 35 is created by the diaphragm member 35 being pushed or depressed toward the housing 70. The diaphragm member 35 is pushed or depressed toward the housing 70 by the oscillation or pushing/pulling motion of the plunger 40 once said plunger 40 has been activated by the motor, as described in more detail below.

FIG. 3 is a partial enlarged cross-sectional view illustrating one embodiment of the disposable pump member 50. Contained within the body of the housing 70 are a first fluid communication channel 90, a one-way inlet valve 20, a one-way outlet valve 25, a second fluid communication channel 95, and power source 45. The arrows represent the direction of the flow of fluid through the device.

The first fluid communication channel 90 provides for open fluid communication between the inlet channel 13 and reservoir 30. Because of the one-way inlet valve 20 the fluid can only travel from the first fluid communication channel 90 through the one-way inlet valve 20 and into the reservoir 30, but not in the other direction. The one-way inlet valve 20 may be a low pressure valve that is commonly known in the art, such as a pressure responsive ball valve or duck bill valve. In this embodiment the pressure range of the inlet valve 20 may be below 10 psi. The purpose of using a low pressure inlet valve 20 is so fluid can be drawn into the reservoir 30 through the small amount of pressure created from the expansion of the diaphragm 35 and the effect of gravity when the diaphragm member 35 moves from a compressed or depressed state to its original dome like shape of highest potential energy. As the fluid travels through the inlet valve 20 it then enters into the reservoir 30. The one-way inlet valve 20 prevents fluid from backflow into fluid communication channel 90 or the fluid source 5.

The second fluid communication channel 95 is a pathway for fluid to travel between the reservoir 30 and the outlet port 10. The second fluid communication channel 95 provides for open fluid communication between the one-way outlet valve 25 and the outlet port 10. The flow of the fluid being delivered, as depicted by arrows, travels from the reservoir 30 through the one-way outlet valve 25 exiting from the outlet port 10. The fluid is mechanically driven from reservoir by a drive mechanism 55 as will be explained in more detail below.

An additional benefit of the device is the ability to manufacture disposable pump members 50 with different pre-determined outlet valve 25 pressures. The disposable pump member 50 may be manufactured with any one of the wide range of pre-determined outlet valve 25 pressures that are currently known in the art, such as a pressure responsive ball valve or duck bill valve. This allows for the end user to know exactly what rate and pressure the fluid is going to be delivered by selecting a pump member 50 having the desired pressure rating. For example, if an intended treatment requires infusion at a rate of 350 psi, the user would select to use a disposable pump member with the one-way outlet valve 25 of at least a 350 psi rating. It is possible to manufacture disposable pump members 50 with an outlet valve 25 capable of producing a pressure anywhere in 10-800 psi range.

The disposable delivery pump member 50 contains a power source 45, such as a small disposable battery that is commonly known in the art. The power source 45 is used to activate the motor of the delivery device. The advantage of using disposable batteries as a power source 45 is that the device does not need to be recharged after each use. After the treatment has been completed the pump member 50 may be disconnected from the drive mechanism 55 and then the delivery pump member 50 is disposed of and the delivery mechanism can be sterilized. Alternatively, in another embodiment the disposable power source is in the drive mechanism 55. The purpose of having the disposable power source located in the drive mechanism 55 is that after the treatment has been completed both the drive mechanism 55 and pump member 50 can be disposed of. In yet another embodiment, the drive mechanism 55 has a rechargeable power supply so that after treatment is complete the pump member 50 is disposed of, and the drive mechanism 55 is sterilized and recharged for a future use.

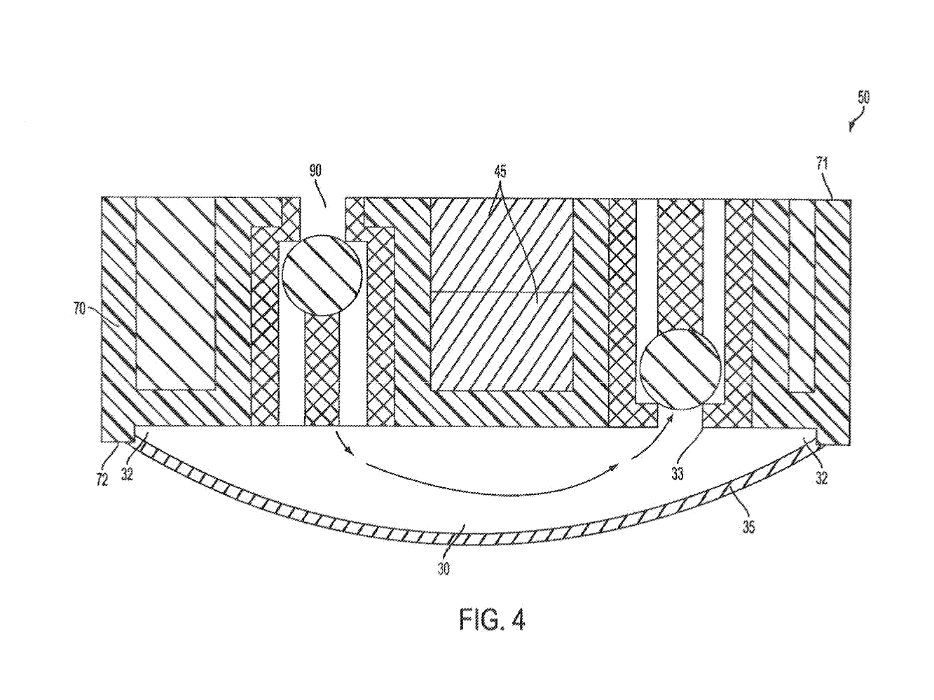

FIG. 4 depicts a partial enlarged cross-sectional view of the housing 70 and pump chamber comprising a diaphragm member 35 and reservoir 30 of the disposable pump member 50. In the illustrated embodiment the reservoir 30 is the space between the bottom surface 72 of the housing 70 and the diaphragm member 35. The bottom surface 72 of the housing may be straight. Alternatively, it is also conceived that the bottom surface 72 has a concave shape toward the top surface 71 of the housing 70.

To control the amount of fluid being delivered, in one embodiment of this invention the entire volume of the reservoir 30 is delivered or infused with each depression of the diaphragm 35—so that the volume of the fluid being delivered is the same, fixed or dependent on the volume capacity of the reservoir 30. Such a method of delivering fluid will aid in delivery of precise amount of fluid for repeated injections.

The reservoir 30 has a pre-determined fluid volume or fluid capacity. The purpose of a reservoir 30 with a pre-determined volume or fluid capacity is that the user will know the exact amount of fluid being infused/delivered with every injection. A benefit of this device is that the user can easily control the volume of fluid to be delivered since each disposable pump member 50 has a pre-determined volume reservoir 30. The volume of the reservoir 30 can range from 1/10 of a milliliter to 10 milliliters.

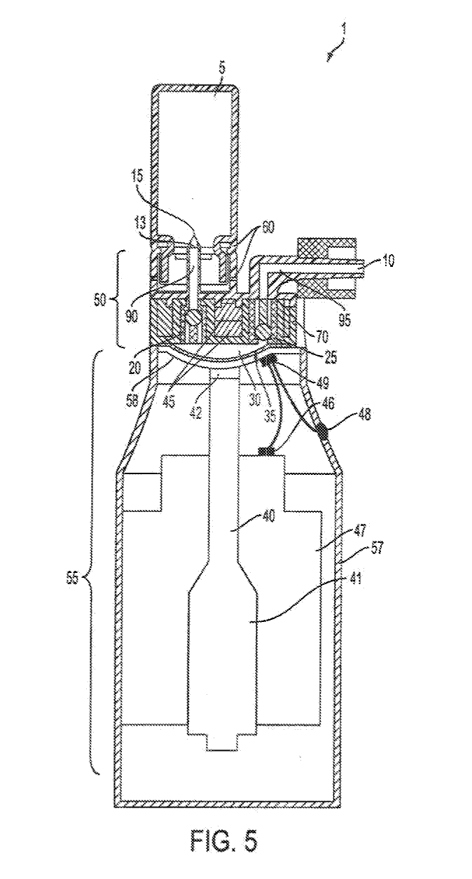

FIG. 5 depicts a cross-sectional view of the self-powered delivery device 1 illustrating the mechanism by which fluid is driven at a controlled pressure. The drive mechanism 55 is comprised of an outer housing 57 containing an elongated plunger element 40 and a plunger activation mechanism 47. The plunger activation mechanism 47 may be either a solenoid (magnet driven) or electric motor which when activated compresses the diaphragm 35 by driving the plunger 40 into contact with the diaphragm 35. Attached to the drive mechanism 55 is a button, trigger or deployment mechanism 48 which when depressed by the user electrically connects the power supply 45 in the disposable pump member 50 to the motor or plunger activation mechanism 47, with common electrical connections such as insulated wiring known in the art, which in turn drives the plunger 40 upward.

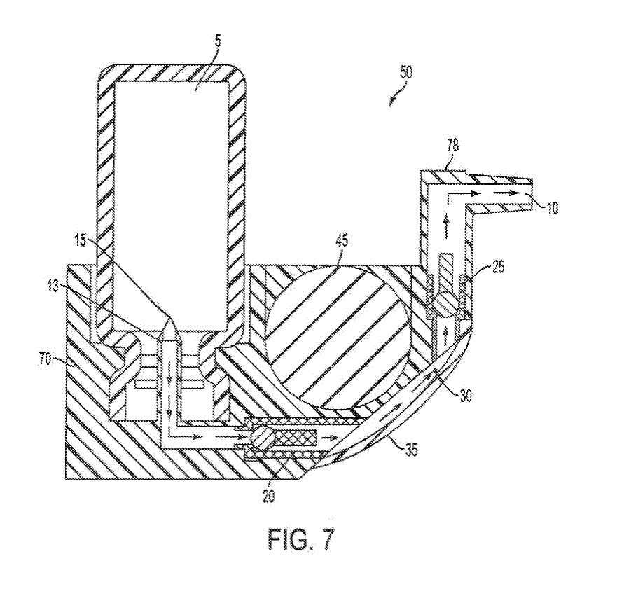

The drive mechanism 55 may be reusable or disposable. The drive mechanism 55 can be made in a number of shapes and sizes, for example in FIG. 5 the drive mechanism 55 is intended to be hand held canister or tube. However, in FIG. 7 the drive mechanism 55 is in the shape of a spray gun or trigger handle. If the drive mechanism 55 is reusable it may be made of durable material that is capable of withstanding multiple uses and may be able to be sterilized between each use. Alternatively, if the drive mechanism 55 is a single-use unit it may be made of a disposable material such as plastic or other similar material. In the illustrated embodiment the activation mechanism 47 is a motor such as a standard motor, prizio motor, or solenoid motor.

The plunger element 40 has a first end 41 that is connected to the activation mechanism 47 and a second end 42 that when activated is in direct contact with the diaphragm 35. Once the fluid source 5 has been connected to the disposable pump member 50 the device will be primed by activating the plunger element 40 causing contact with and depression of the diaphragm 35 and forcing any trapped air to be ejected or expelled from the pump chamber or reservoir 30. Thus, as the user primes the device any trapped air will be forced from the reservoir 30 through the second fluid communication channel 95 to be ejected from the device through outlet port 10. Alternatively, the drive mechanism 55 may have a resilient concave member 58 that is securely attached to the drive mechanism 55 and located between the second end 42 of the plunger 40 and the diaphragm member 35. If such a resilient concave member 58 is used then when the plunger 40 is driven toward the housing 70 the resilient concave member 58 is directly contacted by the second end 42 of the plunger 40 and in turn will be deformed and pushed into a second convex shape toward the housing and physically contacts and depresses the diaphragm member 35. When the second end 42 of the plunger 40 is then pulled away from the housing 70 and is no longer in contact with the resilient concave member 58 then both the diaphragm member 35 and resilient concave member will return to their original shape.

If the drive mechanism is intended to be reusable then the activation mechanism 47 and plunger element 40 may be made of reusable materials commonly known in the art, such as plastics or metals. When a deployment mechanism 48 is depressed or activated it completes a power connection or circuit between the power source 45 and activation mechanism 47. The electrical connection 49 between the deployment mechanism 48 and activation mechanism 47 or motor is dependant on the location of the power source 45. For example, if the power source 45 is located in the pump member 50 then electrical connections must be made between the pump member and the drive mechanism 55 in order to complete the circuit. However, if the power source 45 is contained within the drive mechanism, such as a reusable or rechargeable battery, then all of the electrical circuitry may be self-contained within the drive mechanism 55. Once the electrical connection between the power source 45 and activation mechanism 47 is complete, the activation mechanism 47 is sufficiently powered and drives, forces, or pushes the plunger element 40 upward into contact with the diaphragm member 35. As the plunger element 40 drives towards and compresses the diaphragm member 35 this force will depress or compress the diaphragm member 35 creating enough pressure in the reservoir 30 to crack or open the one-way outlet valve 25. One the one-way outlet valve 25 has been cracked or opened the fluid present in the reservoir 30 is forced through the open outlet valve 25 into the second fluid connection channel 95 and out the outlet port 10 and into the delivery means. Thus, the rate, force, velocity, and speed at which the plunger element 40 is driven and compresses the diaphragm 35 creates a pressure differential sufficient to open or crack the outlet valve 25 and force fluid out of the pump. Once the cracking or open pressure has been reached within the reservoir 30 any further compression of the diaphragm member 35 will result in all fluid within the reservoir 30 to be forced through the second fluid communication channel 95 and port outlet port 10.

Alternative embodiments use an activation mechanism 47 that does not require a power source. Such alternative activation mechanisms include compressed gas, pump, motor, or a spring to drive the plunger element 40 toward the diaphragm 35. In these alternative embodiments the activation mechanism 47 may drive the plunger element 40 in a direction toward the housing 70 causing the diaphragm member 35 to depress or compress. The required force and rate necessary for the plunger element 40 to depress or compress the diaphragm member 35 and force the fluid to exit the pump is directly related to and dependent on the cracking pressure or valve open pressure of the outlet valve 25 used.

Alternatively, it is envisioned that the volume of the fluid being delivered can be less than the total volume of the reservoir 30. In this alternate embodiment a control mechanism (not shown), as currently known in the art, is attached to the drive mechanism 55 and controls the movement of the plunger element 40. The control mechanism is used to select, control, or alter the volume of fluid that is to be delivered. The volume change in the reservoir 30 has a direct relation to the amount of fluid pumped and is controlled by the depth or amount the plunger element 40 compresses or depresses the diaphragm member 35. For example, the further the plunger element 40 drives into and further compresses the diaphragm member 35 the greater the volume change in the reservoir 30 and more fluid being delivered. The control mechanism may have pre-set distances of how far the activation mechanism 47 should drive the plunger element 40 and compress the diaphragm 35. Thus, these pre-set distances will directly relate to the total volume change in the reservoir 30 and the amount of fluid delivered. For example, the volume of the reservoir 30 may have a maximum of 5 milliliters. By controlling, selecting, or altering the volume change in the reservoir 30 with the control mechanism the user can control the activation mechanism 47 so that the plunger element 40 depresses the diaphragm 35 such a distance so that the reservoir 30 has a volume change so that only 3 milliliters of fluid is delivered.

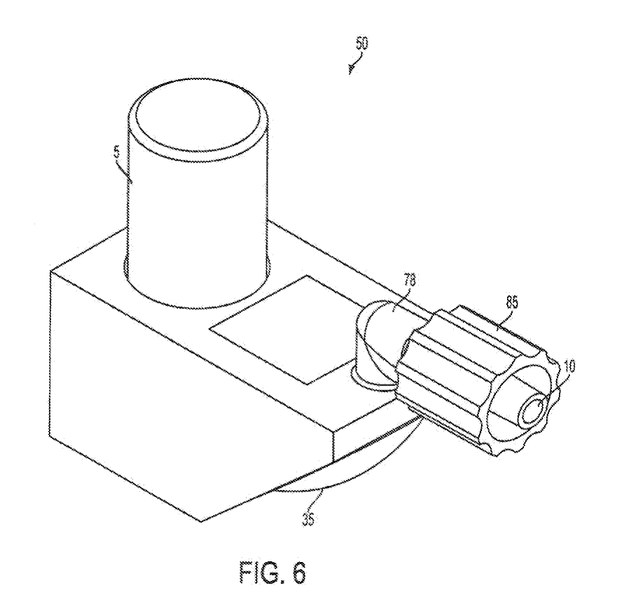

FIGS. 6-8 illustrate an alternative embodiment of the disposable pump member 50. FIG. 6 is an isometric view of a second embodiment of the disposable pump member 50 of the self-powered infusion device. In this embodiment, the housing 70 has a unique shape that is intended to specifically fit and be inserted into a compact drive mechanism 55 shaped like a gun or spray handle as seen in FIG. 8. As seen in FIG. 6 the diaphragm is no longer on the bottom surface of the housing 70 but rather on an angular side. The placement of the diaphragm 35 is important since it must be in direct contract with the plunger element.

As seen in FIG. 7, the pump member 50 of the second embodiment has all of the same components of the first embodiment. The piercing member 15 is inserted into a fluid source 5. Fluid from the fluid source will travel through the inlet channel 13 and into the first fluid communication channel 90. The one-way inlet valve 20 has similar pressure ratings as the first embodiment and may be made from known one-way valves in the art. The diaphragm member 35 is made from a flexible and resilient material that has a preformed convex dome shape. The space between the diaphragm member 35 and the housing 70 is the reservoir 30. However, unlike the first embodiment in this second embodiment the diaphragm member 35 is located on a side wall 73 of the housing 70. Alternatively, it is also conceivable that the second embodiment could also have the diaphragm member 35 located on different walls of the housing 70 such as the other side wall or the bottom surface 72 of the housing 70. The second embodiment may also have disposable power source 45 located in the housing 70, or as described above, the power source may be located in the drive mechanism 55 and may be disposable or rechargeable. Also, the power source 45 of the second embodiment may be easily accessible to the user at the top of the housing 70. For example, if disposable batteries known in the art are used as the power source 45 the user may have easy access to these batteries from the top of the housing 70 with a lid or covered so when the batteries die or lose their power the user may easily exchange or change the batteries. The surface of the housing enclosed by the diaphragm member 35 will have a concave surface so that the highest open point of the reservoir 30 is at the one-way outlet valve, and this allows for all air to be expelled out of the reservoir 30 during use. The outlet valve 25 has similar pressure ratings as the first embodiment and may be made from known one-way valves in the art.

FIG. 8 depicts a compact drive mechanism 55 of the second embodiment in the shape of a gun or spray handle. The advantage of using a compact delivery device 55 in the shape of a gun or spray handle is so the user may have a better grip on the device and it is more comfortable during use. Also, by using a compact delivery device 55 that is smaller in size this will allow for less materials to be required for manufacturing the device. The drive mechanism of the second embodiment contains a deployment mechanism 48, a plunger activation mechanism 47 and a plunger 40 that is intended to contact and depress the resilient diaphragm member 35. The deployment mechanism 48 is electrically connected to the plunger activation mechanism through standard electrical connections known in the art. The delivery means (not shown), such as a catheter, would be attached to the outlet port 10.

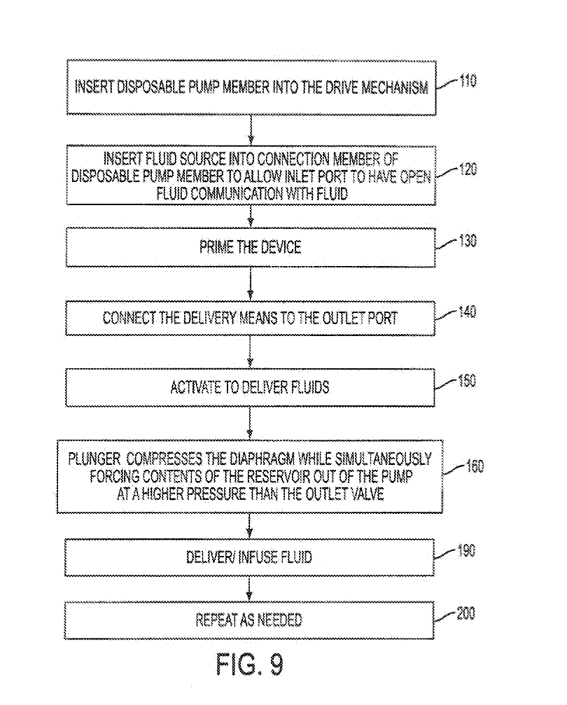

In operation, as depicting in FIG. 9, the first step 110 is for the user to insert the disposable pump member 50 into the drive mechanism 55. Next, the fluid source 5 may be inserted into the connection member 60 causing the piercing member 15 to break the seal of the fluid source 5 creating open fluid communication 120 between the pump member and fluid source 5. Once the fluid source 5 is connected the device must be primed 130. To prime the device 130 the user depresses the deployment mechanism 48 which in turn completes the power connection between the power supply 45 and the activation mechanism 47 or motor. Once the activation mechanism 47 or motor is powered it drives, forces, or pushes the plunger element 40 in the direction toward the housing 70 compressing (not shown) the diaphragm 35. By sufficiently compressing the diaphragm member 35 to reach the cracking or opening pressure of the outlet valve 25 any air trapped in the reservoir 30 will be forced through the open outlet valve 25 into the outlet channel 95 and exit through the outlet port 10. The activation member 47 will then drive the plunger element 40 in the direction away from the housing 70. As the drivable pump 40 moves away from the housing 70 this allows the diaphragm 35 to return to its resiliently biased point of lowest potential energy in which it takes a cup, convex or dome shape. As the diaphragm 35 expands to its resiliently biased point of lowest potential energy this will create enough pressure to crack or open the inlet valve 20 and fluid will move from the area of highest pressure (fluid source 5) to the area of lowest pressure (reservoir 30). Thus, the higher pressure of the fluid source 5 forces fluid to enter the inlet port 13, travel along the inlet channel 90, move through the inlet valve 20 and fill the reservoir 30. Because the pressure differential between the fluid source 5 and reservoir 30 is low, the inlet valve 20 is designed to be a low pressure valve to facilitate the fluid to fill the reservoir 30. Once the reservoir 30 is filled with fluid the device is primed 130 and ready for use.

The user positions the delivery means in the desired anatomical location and then connects 140 the proximal end of the delivery means to the outlet port 10 as previously described. When activation of device for fluid delivery 150 is desired, the deployment mechanism 48 is depressed causing a power connection between the power supply 45 and activation member 47 to activate. Once the device is activated to delivery fluids 150 the plunger element 40 is driven towards the housing 70 forcing the diaphragm 35 to compress with enough force to create a pressure greater than the outlet valve 25, also known as the cracking point of the outlet valve 25. Once the pressure within the reservoir reaches a pressure greater than the outlet valve 25 pressure, fluid is driven from the reservoir 160 through the outlet valve 25 into the outlet channel 95. The fluid exits through the outlet port 10 into the delivery means (not shown) delivering or infusing fluid 190 to the treatment site. Thus, the pressure that the fluid is infused or delivered is dependent on the pressure limit of the outlet valve 25, or the cracking point. Repeat the activation of the device 150 as needed 200 until the treatment is complete.

1. A self-powered infusion pump comprising:
a pump housing;
an inlet channel in fluid communication with an inlet valve for allowing fluid to flow between a fluid source and a pump chamber;
the pump chamber comprising a dome reservoir and convex dome diaphragm being resiliently biased towards a selected first expanded shape and first volume that is movable towards a second depressed shape and second volume;
an outlet valve for selectively allowing fluid to flow between the dome reservoir and an outlet port; and
an outlet channel in fluid communication with the outlet valve for allowing fluid to flow between the pump chamber and the outlet port.

2. The self-powered infusion pump of claim 1, further comprising a power source.

3. The self-powered infusion pump of claim 2, wherein the pump housing is inserted into a drive mechanism.

4. The self-powered infusion pump of claim 3, wherein the drive mechanism contains a motor and a deployment mechanism.

5. The self-powered infusion pump of claim 4, wherein the deployment mechanism is electrically connected to the power source and the motor.

6. The self-powered infusion pump of claim 5, wherein the inlet valve is a one-way valve having a pressure rating up to 10 pounds per square inch.

7. The self-powered infusion pump of claim 6, wherein the outlet valve is a one-way valve having a pressure rating up to 800 pounds per square inch.

8. The self-powered infusion pump of claim 7, wherein the fluid is a sclerosing agent.

9. A method of infusing fluid into a hollow anatomical structure at a controlled high pressure using a self-powered infusion pump comprising:
inserting a pump housing into a delivery device containing a motor and a plunger connected to the motor, the pump housing comprising:
an inlet channel in fluid communication with an inlet valve for allowing fluid to flow between a fluid source and a pump chamber;
the pump chamber comprising a dome reservoir and convex dome diaphragm being resiliently biased towards a selected first expanded shape and first volume that is movable towards a second depressed shape and second volume;
an outlet valve for selectively allowing fluid to flow between the dome reservoir and an outlet port; and
an outlet channel in fluid communication with the outlet valve for allowing fluid to flow between the pump chamber and the outlet port;
attaching the inlet channel into a fluid source;
attaching the proximate most end of a delivery means to the outlet port;
powering the motor of the delivery device with a power source;
compressing the dome reservoir towards the second depressed shape and second volume with the plunger;
expanding the dome reservoir back to the first expanded shape and first volume causing an increase in internal pressure and fluid to travel though the inlet valve and fill the dome reservoir.

10. A method of infusing fluid into a hollow anatomical structure at a controlled high pressure using a self-powered infusion pump of claim 9, further comprising:
delivering the fluid through the outlet valve at pressures up to 800 pounds per square inch.

11. A method of infusing fluid into a hollow anatomical structure at a controlled high pressure using a self-powered infusion pump of claim 9, further comprising:
delivering a sclerosing agent as the fluid source.

12. A method of infusing fluid into a hollow anatomical structure at a controlled high pressure using a self-powered infusion pump of claim 9, further comprising:
disposing of the power source upon completion of use.

13. A method of infusing fluid into a hollow anatomical structure at a controlled high pressure using a self-powered infusion pump of claim 9, further comprising:
recharging the power source upon completion of use.
